(19) United States
(12) Patent Application Publication
(10) Pub. No.: US 2018/0092594 A1
(43) Pub. Date: Apr. 5, 2018

(54) FAT TISSUE OBSERVATION METHOD

(71) Applicants: OLYMPUS CORPORATION, Tokyo (JP); SARATOV State University, Saratov (RU)

(72) Inventors: Yohei Tanikawa, Tokyo (JP); Yasunobu Iga, Tokyo (JP); Shinichi Takimoto, Tokyo (JP); Valery V. Tuchin, Saratov (RU); Elina A. Genina, Saratov (RU); Alexey N. Bashkatov, Saratov (RU); Irina Yu. Yanina, Saratov (RU); Yana V. Tarakanchikova, Saratov (RU); Georgy S. Terentuk, Saratov (RU); Polina A. Timoshina, Saratov (RU); Daria K. Tuchina, Saratov (RU)

(73) Assignees: OLYMPUS CORPORATION, Tokyo (JP); SARATOV State University, Saratov (RU)

(21) Appl. No.: 15/831,675

(22) Filed: Dec. 5, 2017

Related U.S. Application Data
(63) Continuation of application No. PCT/JP2016/067297, filed on Jun. 3, 2016.

(30) Foreign Application Priority Data
Jun. 11, 2015 (RU) 2015122756

(51) Int. Cl. A61B 5/00

(52) U.S. Cl. CPC A61B 5/489; A61B 2505/05; A61B 5/0086; A61B 5/4872

(57) ABSTRACT

A fat tissue observation method includes: a process A of applying an optical clearing agent to fat tissue within a body; a process B of heating the fat tissue to reduce a first refractive index of the fat tissue and put the first refractive index close to a second refractive index of the optical clearing agent; and a process C of observing an inside of the fat tissue that the optical clearing agent permeates.

This application is a continuation application based on a PCT Patent Application No. PCT/JP2016/067297, filed on Jun. 3, 2016, whose priority is claimed on Russian Patent Application No. 2015122756, filed on Jun. 11, 2015. The contents of both the PCT Application and the Japanese Application are incorporated herein by reference.

### TECHNICAL FIELD

The present invention relates to a fat tissue observation method.

### BACKGROUND ART

In the related art, when a surgical site is covered with fat tissue during a surgery, it is necessary to remove the fat tissue in order to treat the surgical site. When this fat removal treatment is done, it is preferable to confirm the position of a blood vessel that runs inside or under the fat tissue to be removed. In relation to such a purpose, an imaging system capable of visually recognizing the running state of a blood vessel at a deep portion of a body during a surgery is known (for example, refer to Japanese Unexamined Patent Application First Publication No. 2007-75445).

The imaging system described in Japanese Unexamined Patent Application First Publication No. 2007-75445 includes a light source device that illuminates a body with infrared light, and an infrared camera that detects the light reflected from the body illuminated with this light source. The illuminated light from the light source device has a wavelength of the infrared region in which light-scattering properties of the fat tissue are low. For this reason, since the illuminated light is not strongly scattered by the fat tissue, the illuminating light penetrates up to a deep portion of the fat tissue. Additionally, the infrared camera includes a wavelength band limited filter, an imaging lens, and an image sensor. The light reflected in the body is focused on the image sensor by the imaging lens via the wavelength band-pass filter. Additionally, the wavelength range of the wavelength band limited filter is set so that the filter transmits the light with wavelength where the absorption coefficient of the blood vessel becomes a local maximum. This enables the blood vessel of the deep portion of the body to be visually recognized.

### SUMMARY OF INVENTION

A fat tissue observation method related to a first aspect of the present invention includes a process A of applying an optical clearing agent to fat tissue within a body, a process B of heating the fat tissue to reduce a first refractive index of the fat tissue and put the first refractive index close to a second refractive index of the optical clearing agent, and a process C of observing an inside of the fat tissue that the optical clearing agent permeates.

The fat tissue observation method related to a second aspect of the present invention may further include a process D of heating the optical clearing agent to a predetermined temperature.

In the fat tissue observation method related to a third aspect of the present invention, the process D may be performed before the process A.

In the fat tissue observation method related to a fourth aspect of the present invention, the process A may have the process D of heating the optical clearing agent to a predetermined temperature, and the optical clearing agent may be heated to the predetermined temperature by a heating unit provided at a tip portion of an optical clearing agent supply path in which the optical clearing agent is applied to the fat tissue, and the heated optical clearing agent is applied to the fat tissue.

In the fat tissue observation method related to a fifth aspect of the present invention, in the process B, a temperature of the fat tissue to which the optical clearing agent is applied may be measured, and a temperature of the optical clearing agent is controlled so that the difference between the temperature of the fat tissue and a predetermined target temperature becomes small.

### BRIEF DESCRIPTION OF THE DRAWINGS

FIG. 1 is a view shown one process of a fat tissue observation method related to the first embodiment.

FIG. 2 is a view schematically shown the refractive index distribution of a fat tissue related to the first embodiment.

FIG. 3 is a view schematically shown changes in the refractive index distribution of the fat tissue related to the first embodiment.

FIG. 4 is a view shown one process of the fat tissue observation method related to the first embodiment.

FIG. 5 is a view schematically shown changes in the refractive index distribution of the fat tissue related to the first embodiment.

FIG. 6 is a view shown one process of the fat tissue observation method related to the first embodiment.

FIG. 7 is a view shown one process of a fat tissue observation method related to a second embodiment.

FIG. 8 is a view shown the temperature of an optical clearing agent related to the second embodiment.

FIG. 9 is a view shown one process of a fat tissue observation method related to a third embodiment.

FIG. 10 is a view shown one process of a fat tissue observation method related to a fourth embodiment.

### DESCRIPTION OF EMBODIMENTS

### First Embodiment

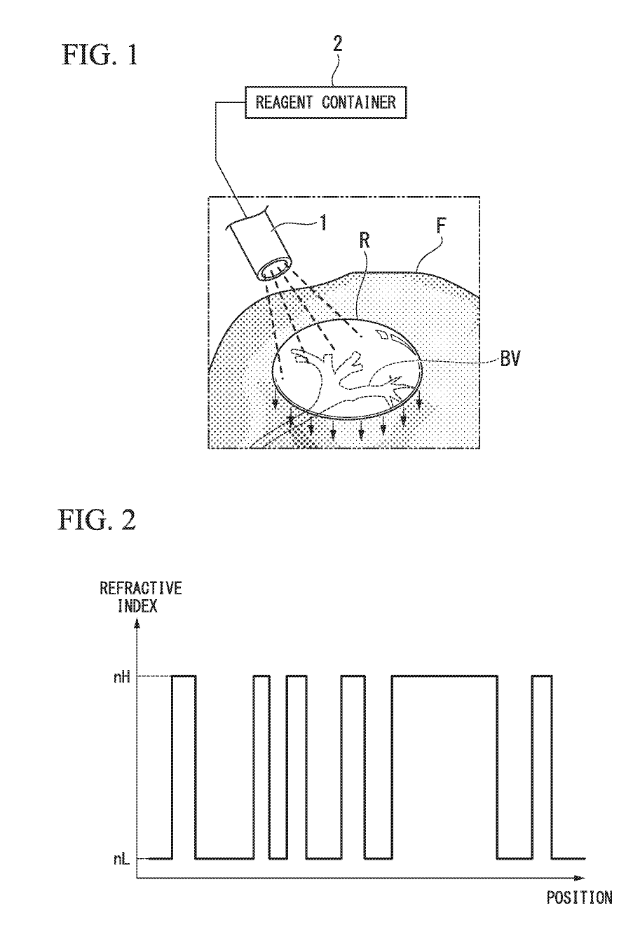

A fat tissue observation method related to a first embodiment of the present invention will be described with reference to FIGS. 1 to 6.

In the fat tissue observation method of the present embodiment, for example, as illustrated in FIG. 1, a spraying unit equipped with a tube (optical clearing agent supply path) 1 that sprays an optical clearing agent R to fat tissue F, and a reagent container 2 that is provided outside a body and contains the optical clearing agent R is used. The tube 1 is connected to the reagent container 2, and enables the optical clearing agent R within the reagent container 2 to be supplied to the fat tissue F via the tube 1.

Additionally, the “optical clearing” of the optical clearing agent related to the present invention means that light permeability obtained by using light of at least a portion of wavelengths is improved, or the scattering properties obtained by using light of at least a portion of wavelengths are decreased.

The optical clearing agent used in the present embodiment is a compound having a specific structure, and can make biological tissue cleared optically by means of the permeation of optical clearing agent to biological tissue. In the present embodiment, the optical clearing agent is not particularly limited if biological tissue can be cleared optically by the optical clearing agent. For example, saccharides, such as sucrose and glucose, can be preferably used. Additionally, the optical clearing agent is not limited to saccharides, and polyethylene glycols or the like can also be used.

Generally, as illustrated in FIG. 2, the fat tissue F is configured such that a low refractive index region (hereinafter referred to as a low refractive index portion) having a refractive index nL, and a high refractive index region (hereinafter referred to as a high refractive index portion: first refractive index) having a refractive index nH higher than the refractive index nL are present in a mixed manner.

(Process A)

First, a situation that the abdominal cavity can be accessed is created, for example, by incising an abdominal wall or making an opening in an abdominal wall. As illustrated in FIG. 1, the tube 1 is directed to a reagent application site of the fat tissue F, and the optical clearing agent R is applied to the fat tissue F from the reagent container 2. The reagent application site includes fat tissue that needs to be removed, for example, fat tissue that is located on a surgical site, or fat tissue that covers the periphery of a surgical site when the surgical site is buried.

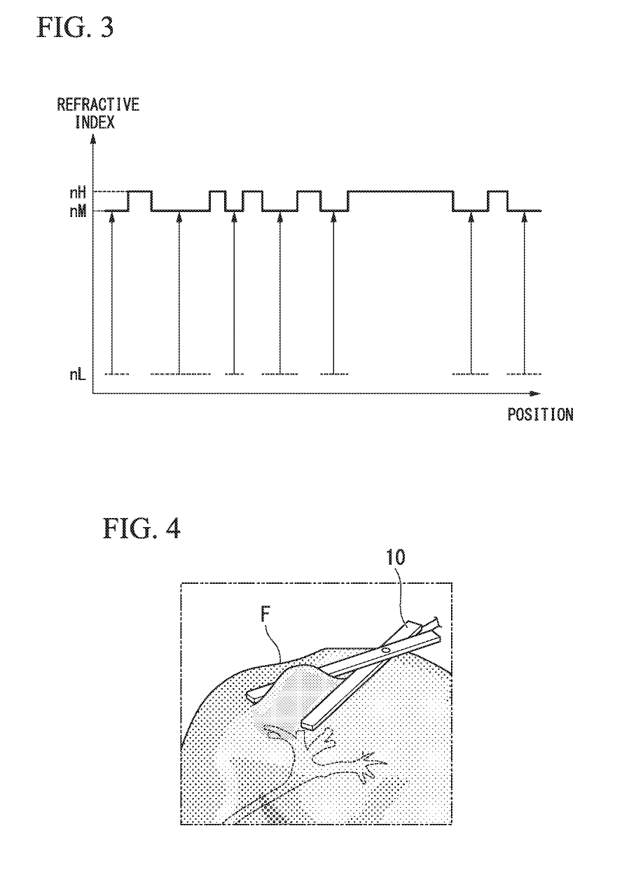

If the optical clearing agent R is applied to the fat tissue F, a body fluid within the fat tissue F, and the optical clearing agent R are replaced with each other. As a result, as illustrated in FIG. 3, the refractive index of the low refractive index portion is increased according to the refractive index (second refractive index) of the optical clearing agent R, and the value thereof reaches nM and approach the refractive index nH. In this case, as illustrated in FIG. 1, the optical clearing agent R begins to permeate the fat tissue F, but the doctor cannot clearly observe a blood vessel BV that runs inside or under the fat tissue F because the permeation depth of optical clearing agent R to fat tissue F is not sufficient.

[Process B]

Next, the tube 1 is taken out from the body, and as illustrated in FIG. 4, a heating device 10 is introduced into the body, and the fat tissue F to which the optical clearing agent R is applied is heated to a temperature equal to or higher than the body temperature (for example, 40° C. to 45° C.). Although the method of heating the fat tissue F is not particularly limited, an example in which the fat tissue F is gripped by means of the heating device 10 of a type in which tissue is pinched is illustrated in the present embodiment. The following three effects are obtained by the fat tissue F being heated.

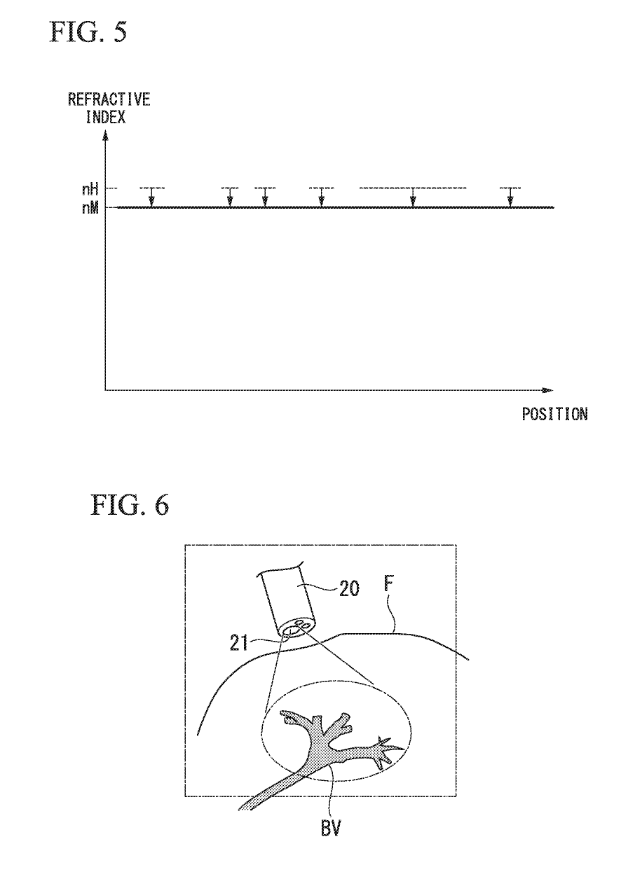

1. As illustrated in FIG. 5, the refractive index of mainly the high refractive index portion in the fat tissue is decreased, and this value approaches the refractive index nM (refractive index of the optical clearing agent R). As a result, the refractive index of the entire fat tissue F becomes substantially uniform.

2. The permeation speed to the fat tissue F of the optical clearing agent R is improved, and the optical clearing agent R can be delivered to a deep portion of the fat tissue F in a shorter time.

3. The degree of light scattering by the fat tissue F becomes lower due to the fluidity improvement of the fat tissue F (due to the phase transition of the fat tissue from a solid state to a liquid state).

[Process C]

Next, the inside of the fat tissue F that the optical clearing agent R has sufficiently permeated is observed. For the observation of the fat tissue F, for example, an endoscope equipment is used. As illustrated in FIG. 6, signals sent through an observation optical system from an observation window 21 of an endoscope equipment 20 are displayed as an observation image on a monitor (not illustrated). The observation image illuminated with illumination light is displayed on the monitor. In this case, since the refractive index of the fat tissue F is substantially uniform, scattering of the illuminating light by fat tissue F is suppressed. Accordingly, the doctor can clearly observe the blood vessel BV that runs inside or under the fat tissue F.

The doctor can easily remove the fat tissue F that needs removal through the process C.

In order to decrease light scattering by fat tissue, it is necessary to make the refractive index of the entire fat tissue as uniform as possible. For that purpose, it is preferable to use an optical clearing agent that satisfies the following two conditions.

1. The optical clearing agent has a refractive index near the refractive index of the high refractive index portion in the fat tissue.

2. The optical clearing agent has a high permeability into the fat tissue.

If the above conditions are taken into consideration, it is preferable that the optical clearing agent has a refractive index of about 1.45 from a viewpoint of the refractive index, and it is preferable that the optical clearing agent has a hydrophilic property from a viewpoint of the permeability. Although solutions (sucrose, glucose, and the like) of saccharide compounds are suitable as candidates for the optical clearing agent that satisfy these conditions, in order to realize a refractive index of about 1.45, the concentration of these solutions should be adjusted near the concentration of saturated solution, and the viscosity of the optical clearing agent becomes consequently high.

Since permeation of the optical clearing agent into the fat tissue is governed by Fick's law, the temperature and the permeation speed have a proportional relationship, and the viscosity and the permeation speed has an inversely proportional relationship. Therefore, higher viscosity of the optical clearing agent leads to the decrease of the permeability to the fat tissue. In the case of the solutions of the saccharide compounds, it is preferable that the refractive index is adjusted to about 1.41 to about 1.43 if the balance between a high refractive index and low viscosity are taken into consideration. However, in this case, since some deviation occurs between the refractive index of the optical clearing agent and the refractive index of the high refractive index portion of the fat tissue, scattering of light by the fat tissue cannot be made sufficiently low.

Thus, by heating the fat tissue, the above problem can be solved, and scattering of light by the fat tissue can be made sufficiently low, and the permeability of the optical clearing agent to the fat tissue can be improved.

First, if the fat tissue is heated, the refractive index of mainly the high refractive index portion (the portion of the refractive index nH) in the tissue can be lowered. Thus, even if a solution of a saccharide compound having a refractive index of about 1.41 to 1.43 of which the balance between the high refractive index and the low viscosity required for the optical clearing agent is taken into consideration is used, the effect of suppressing the scattering of light can be sufficiently obtained.

Second, if the fat tissue is heated, the optical clearing agent is also heated, and the viscosity of the optical clearing agent decreases. The permeation of the optical clearing agent into the fat tissue can be improved by the effects of both the temperature increase and viscosity decrease of the optical clearing agent.

Additionally, if the fat tissue is heated, the phase transition of the fat tissue from a solid state to a liquid state takes place. Therefore, the degree of light scattering by the fat tissue F becomes lower due to the fluidity improvement of the fat tissue F.

An example regarding the above effect of combining the heating of the fat tissue and the optical clearing agent, thereby suppressing the scattering of light caused by the fat tissue, is illustrated.

As an optical clearing agent to be used, a sucrose solution having a concentration of about 55% is assumed. The refractive index of this optical clearing agent is about 1.43 (the temperature of the optical clearing agent is 20° C. and the wavelength of light is 589.29 nm), and a change in the refractive index with respect to the temperature is about −1.6×10⁻⁴° C.⁻¹. Additionally, the refractive index of the fat tissue is about 1.45 (the temperature of the fat tissue is 20° C. and the wavelength of light is 589.29 nm), and a change in the refractive index with respect to the temperature is about −5.3×10⁻⁴° C.⁻¹.

If the optical clearing agent is applied to the fat tissue, and the temperature in a non-heated situation is assumed to be 30° C., the refractive index difference between the optical clearing agent and the fat tissue reaches about 0.016. If the fat tissue and the optical clearing agent are heated to 45° C. in this state, the refractive index difference between the optical clearing agent and the fat tissue reaches about 0.010, and this difference decreases down to about the half of that before the heating.

Since the deviation between the refractive index of the optical clearing agent and the refractive index of the high refractive index portion of the fat tissue can be made small by combining the heating of the fat tissue and the optical clearing agent in this way, the scattering of light caused by the fat tissue can be decreased.

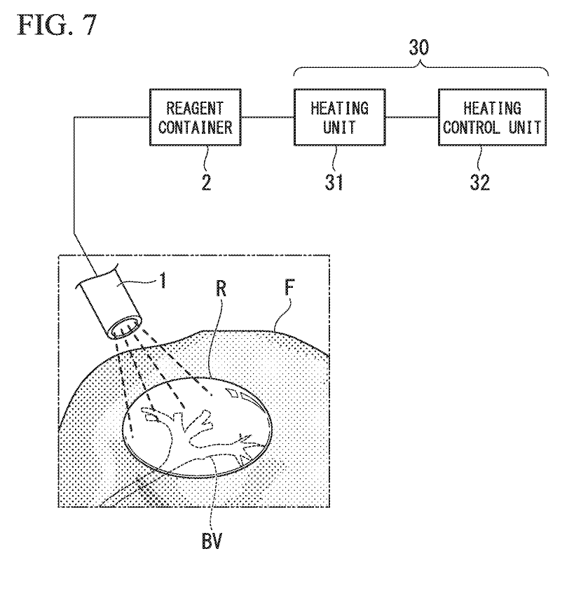

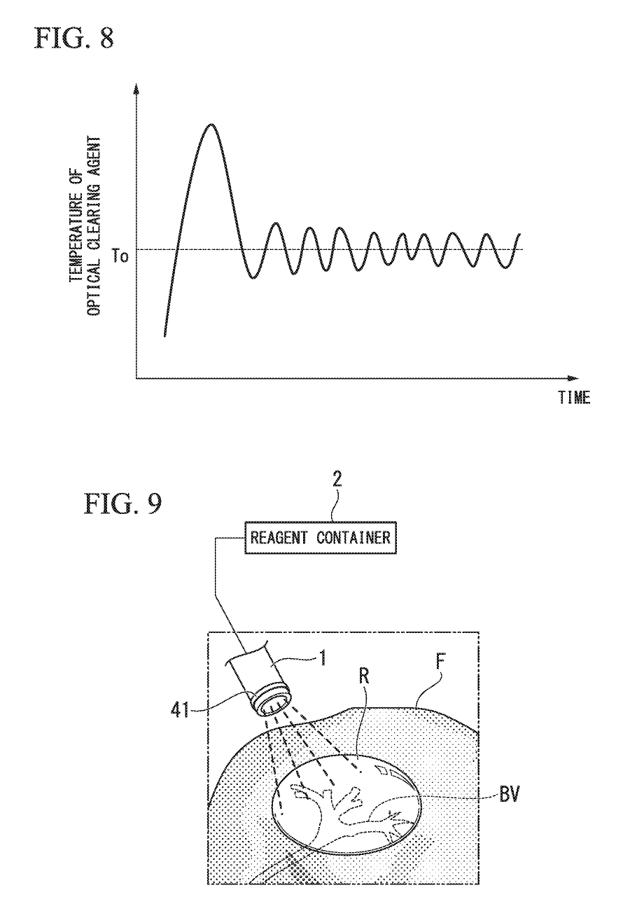

A second embodiment of the present invention will be described with reference to FIGS. 7 and 8.

A fat tissue observation method of the present embodiment is different from that of the first embodiment in that a process of heating an optical clearing agent to a predetermined temperature is performed before the optical clearing agent is applied to fat tissue.

In the following description, constituent elements common to those already described will be designated by the same reference signs, and duplicate description will be omitted.

In the present embodiment, the fat tissue F is not heated by the heating device 10, but the optical clearing agent R is heated using a heating device 30.

The heating device 30 that heats the optical clearing agent used in the present embodiment to a target temperature (predetermined temperature) will be described. The heating device 30, as illustrated in FIG. 7, includes a heating unit 31, and a heating control unit 32. Both of the heating unit 31 and the heating control unit 32 are provided outside a body.

The heating unit 31 is connected to the reagent container 2, and is able to heat the reagent container 2, thereby heating the optical clearing agent R.

The heating control unit 32 is connected to the heating unit 31. The heating control unit 32 controls the preset temperature of the heating unit 31 to be a temperature (for example, 50° C.) higher than a target temperature To for a constant time. Thereafter, the heating control unit performs a control so that the preset temperature of the heating unit 31 is periodically repeated within a predetermined range (for example, 40° C. to 45° C.). As a result, as illustrated in FIG. 8, the temperature of the optical clearing agent R converges on the target temperature To.

Here, the “target temperature” means a desired temperature of the optical clearing agent R when the optical clearing agent R is applied to the fat tissue F.

Next, the flow of the fat tissue observation method in the present embodiment will be described.

In the present embodiment, the process D of heating the optical clearing agent to the target temperature is performed before the above-described the process A.

Process D

First, as above-described, the temperature of the optical clearing agent R in the reagent container 2 is heated by the heating control unit 32 of the heating device 30.

Process A

Next, the optical clearing agent R heated from the reagent container 2 is applied to the fat tissue F via the tube 1. In this case, the optical clearing agent R of the target temperature is applied to the fat tissue F.

Process B

Next, the fat tissue F is heated by the heated optical clearing agent R. The refractive index of the low refractive index portion is increased by the optical clearing agent R, and the refractive index of the high refractive index portion is decreased by heating. As a result, as illustrated in FIG. 5, the entire fat tissue has the refractive index nM.

Process C

Since the refractive index of the fat tissue F is substantially uniform, scattering of the illumination light is suppressed. Accordingly, as illustrated in FIG. 6, the doctor can clearly observe the blood vessel BV that runs inside or under the fat tissue F. Then, the fat tissue F that needs removal is removed.

According to the fat tissue observation method of the present embodiment, the heated optical clearing agent R is applied to the fat tissue F. Accordingly, since the fat tissue F is heated simultaneously with the application of the heating of the optical clearing agent R, it is not necessary to heat the fat tissue F using the heating device 10, and it is possible to perform surgery more rapidly.

Additionally, since the optical clearing agent R is heated, the viscosity of the optical clearing agent R decreases. The permeation of the optical clearing agent R into the fat tissue F can be improved by the effects of both the temperature rise and viscosity decrease of the optical clearing agent.

Moreover, since the optical clearing agent R heated to the target temperature is applied to the fat tissue F, it is possible to certainly make a heated region and a reagent application region coincide with each other. Accordingly, regions that are not an object to be observed are not heated, and an invasion to the tissue can be reduced.

In addition, in the present embodiment, the heating control unit 32 is provided to control the heating unit 31 so that the optical clearing agent R is maintained at a constant temperature. However, the heating control unit 32 is not necessarily provided. In this case, for example, the heating unit 31 may be set to the target temperature.

Additionally, the temperature of the optical clearing agent R may fall depending on surgery environments while the optical clearing agent R sprayed from the reagent container 2 passes through the tube 1. In this case, the preset temperature of the heating unit 31 may be set to be higher than the target temperature, taking into consideration a fall in the temperature of the optical clearing agent R.

Additionally, in the present embodiment, the process D is performed before the process A. However, the optical clearing agent R may be heated separately from the heating of the fat tissue F after the process A.

A third embodiment of the present invention will be described with reference to FIG. 9.

A fat tissue observation method of the present embodiment is different from that of the first embodiment in that the process of applying the optical clearing agent to the fat tissue has the process of heating the optical clearing agent to a predetermined temperature.

In the following description, constituent elements common to those already described will be designated by the same reference signs, and duplicate description will be omitted.

A heating unit 41 that heats the optical clearing agent in the present embodiment to the target temperature (predetermined temperature) will be described.

The heating unit 41 of the present embodiment is provided at a tip portion of the tube 1. The heating unit 41 heats the tip portion of the tube 1 so that the optical clearing agent R has the target temperature.

Next, the fat tissue observation method in the present embodiment will be described.

Process A and Process D

First, the optical clearing agent R is applied to the fat tissue F via the tube 1 from the reagent container 2. In this case, since the optical clearing agent R that has passed through the tip portion of the tube 1 is heated by the heating unit 41, the optical clearing agent R heated to the target temperature is applied to the fat tissue F. That is, the process A of applying the optical clearing agent R to the fat tissue F, and the process D of heating the optical clearing agent R to the target temperature are substantially simultaneously performed.

The process B and the process C are the same as those of the second embodiment.

According to the fat tissue observation method of the present embodiment, the optical clearing agent R heated to the target temperature is applied to the fat tissue F. Accordingly, since the fat tissue F is heated simultaneously with the application of the heating of the optical clearing agent R, it is not necessary to heat the fat tissue F using the heating device 10, and it is possible to perform surgery more rapidly.

Additionally, since the optical clearing agent R is heated by the tip portion of the tube 1, the optical clearing agent R can prevent the temperature of the optical clearing agent R from dropping while passing through the tube 1. As a result, it is possible to prevent the optical clearing agent R from deviating from the target temperature. Therefore, the preset temperature of the heating unit 41 can be the target temperature.

In addition, similar to second embodiment, the heating control unit 32 may be connected to the heating unit 41, and the heating control unit 32 may control the heating unit 41 to keep the temperature of the optical clearing agent R constant.

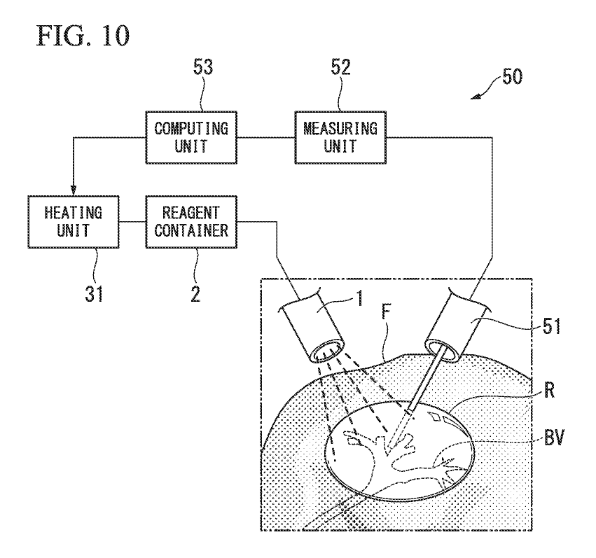

A fourth embodiment of the present invention will be described with reference to FIG. 10.

The fat tissue observation method of the present embodiment is different from the second embodiment in that the temperature of fat tissue where an optical clearing agent is applied is measured.

In the following description, constituent elements common to those already described will be designated by the same reference signs, and duplicate description will be omitted.

The heating device 30 that heats the optical clearing agent in the present embodiment to the target temperature is the same as that of the second embodiment. The heating device 30 is provided with a feedback unit 50.

The feedback unit 50 of the present embodiment includes a temperature measuring instrument 51, a measurement unit 52, and a computing unit 53, all of which are provided outside a body.

The temperature measuring instrument 51 is an instrument that has a tip inserted into the fat tissue F so as to measure the temperature of the fat tissue F, and is inserted into the body at the laparotomy.

The measurement unit 52 is connected to the temperature measuring instrument 51, and the temperature measured by the temperature measuring instrument 51 is input to the measurement unit.

The computing unit 53 compares the temperature of the fat tissue F input to the measurement unit 52 with the target temperature, for example, 40° C. As a result of the comparison, when the temperature of the fat tissue F is lower than the target temperature, the computing unit 53 performs a control so that the preset temperature of the heating unit 31 is higher the current preset temperature.

Next, the fat tissue observation method in the present embodiment will be described.

First, the process D and the process A are performed similar to the second embodiment. After the heated optical clearing agent R is applied to the fat tissue F, in the process B, the temperature of the fat tissue F is measured by the temperature measuring instrument 51. Next, in the computing unit 53, the temperature of the fat tissue F input to the measurement unit 52 is compared with the target temperature. As a result of the comparison, when the temperature of the fat tissue F is lower than the target temperature, the computing unit 53 performs a control so that the preset temperature of the heating unit 31 is higher the current preset temperature. In this way, the computation using the feedback unit 50 is repeated and the temperature of the optical clearing agent R is controlled so that the difference between the temperature of the fat tissue F and the target temperature becomes small.

That is, in the present embodiment, the temperature control of the optical clearing agent R after the optical clearing agent R is applied to the fat tissue F belongs to the process B.

According to the fat tissue observation method of the present embodiment, the fat tissue to which the optical clearing agent is applied can be precisely heated to the target temperature by controlling the temperature of the optical clearing agent R so that the difference between the temperature of the fat tissue F and the target temperature becomes small in the process B.

In addition, the process of measuring the temperature of the fat tissue to which the optical clearing agent is applied can also be applied to the first embodiment or the third embodiment. When this process is applied to the first embodiment, the temperature of the heating device 10 may be controlled by the computing unit 53, or when this process is applied to the third embodiment, the preset temperature of the heating unit 41 may be controlled by the computing unit 53.

While the preferred embodiments of the present invention have been described and illustrated above, it should be understood that this is exemplary of the present invention and is not to be considered as limiting. Additions, omissions, substitutions, and other modifications of components can be made without departing from the concept of the present invention. The present invention is not to be considered as being limited by the foregoing description, and is limited only by the scope of the appended claims.

1. A fat tissue observation method comprising:
a process A of applying an optical clearing agent to fat tissue within a body;
a process B of heating the fat tissue to reduce a first refractive index of the fat tissue and put the first refractive index close to a second refractive index of the optical clearing agent; and
a process C of observing an inside of the fat tissue that the optical clearing agent permeates.

2. The fat tissue observation method according to claim 1, further comprising:
a process D of heating the optical clearing agent to a predetermined temperature.

3. The fat tissue observation method according to claim 2,
wherein the process D is performed before the process A.

4. The fat tissue observation method according to claim 1,
wherein the process A has the process D of heating the optical clearing agent to a predetermined temperature, and
wherein the optical clearing agent is heated to the predetermined temperature by a heating unit provided at a tip portion of an optical clearing agent supply path in which the optical clearing agent is applied to the fat tissue, and the heated optical clearing agent is applied to the fat tissue.

5. The fat tissue observation method according to claim 1,
wherein in the process B,
a temperature of the fat tissue to which the optical clearing agent is applied is measured, and
a temperature of the optical clearing agent is controlled so that the difference between the temperature of the fat tissue and a predetermined target temperature becomes small.
